(19) United States
(12) Patent Application Publication
(10) Pub. No.: US 2004/0024532 A1
(43) Pub. Date: Feb. 5, 2004

(54) Method of identifying trends, correlations, and similarities among diverse biological data sets and systems for facilitating identification

(76) Inventor: Robert Kincaid, Half Moon Bay, CA (US)

Correspondence Address: Agilent Technologies, Inc., Legal Department, DL429, Intellectual Property Administration, P.O. Box 7599, Loveland, CO, 80537-0599, US

(21) Appl. No.: 10/209,477

(22) Filed: Jul. 30, 2002

(51) Int. Cl. G06F 19/00; G01N 33/48; G01N 33/50; G01N 31/00

(52) U.S. Cl. 702/19; 702/20

(57) ABSTRACT

System, tools and methods for inspecting very large data sets of microarray, protein array or other large-scale biological experiments along with other relevant supporting data. Widely diverse but related and potentially correlated data (such as gene expression and clinical observations) can be combined to search for meaningful correlations and trends using innate human pattern recognition.

### FIELD OF THE INVENTION

The present invention pertains to software systems and methods for organizing and identifying trends, correlations and other useful relationships among diverse biological data sets.

### BACKGROUND OF THE INVENTION

The advent of new experimental technologies that support molecular biology research have resulted in an explosion of data and a rapidly increasing diversity of biological measurement data types. Examples of such biological measurement types include gene expression from DNA microarray or Taqman experiments, protein identification from mass spectrometry or gel electrophoresis, cell localization information from flow cytometry, phenotype information from clinical data or knockout experiments, genotype information from association studies and DNA microarray experiments, etc. This data is rapidly changing. New technologies frequently generate new types of data.

Understanding observed trends in gene or protein expression often require correlating this data with additional information such as phenotype information, clinical patient data, putative drug treatments dosages, etc. Even when fairly rigorous computational techniques such as machine learning-based clustering or classification schemes are used, the results of these techniques are typically cross-checked with observed phenotypes or clinical diagnoses to interpret what the computational results might mean.

Currently, correlations of the experimental data with types of additional information as exemplified above by manually (i.e., visually) inspecting the additional (e.g., clinical) data and visually comparing it with the experimental data to look for similarities (i.e., correlations) between experimental and observed phenomena. For example, a researcher might notice a highly up or down regulated gene during inspection of a microarray experiment and then explore the available clinical data to see if any observed clinical data correlates with the known function of the gene involved in the microarray experiment. Finding correlations in this manner could be described as a “hit-or-miss” procedure and is also dependent upon the accumulated knowledge of the researcher. Further, the large volumes of data that are generated by current experimental data generating procedures, such as microarray procedures, for example, makes this method of correlating an extremely tedious, if not impossible task.

Efforts have been made in attempting to visualize and discover overall gene expression patterns from large gene expression data sets with little success. For example, scatter plots and parallel coordinate techniques available with Spotfire 4.0 and Spotfire 5.0 were used by Pan in an attempt to identify expressed sequence tags (ESTs) having expression patterns similar to those of known genes. Both the expression patterns of the ESTs as well as those of the known genes were obtained from a data set including melanoma samples and normal (control) samples provided by National Human Genome Research Inistitute (see Pan, Zhijian: “Application Project: Visualized Pattern Matching of Malignant Melanoma with Spotfire and Table Lens”, http//:www.cs.umd.edu/class/spring2001/cmsc838b/Apps/presentations/Zhijian_Pan/. The use of scatter plots was reported to be incapable of managing the complexity of the data set being examined. The use of parallel coordinates with Spotfire 5.0 was more promising, in that it was capable of displaying all thirty-eight experimental conditions on a single page, where similarities in expression patterns could be searched for.

Table Lens was also employed by the same researcher to visualize expression patterns of the ESTs and known genes. However, it was reported that Table Lens was ineffective, and “very difficult” for use in finding matching patterns. Neither Spotfire (4.0 or 5.0) was used to compare expression or other experimental data with supporting clinical data or data sets of any other type, but were only used in attempting to group like data within the experimental data set.

More powerful methods of combining widely diverse, but related and potentially correlated biological data sets are needed to improve the ease, speed and efficiency of correlating information in these data sets. Further, more powerful methods are needed to improve the probability that such correlations will be identified.

### SUMMARY OF THE INVENTION

The present invention provides a system, methods and tools for visually inspecting diverse, very large data sets of biological data to identify trends, correlations or relations among data from the data sets. A method for identifying such trends, correlations or relations may include inputting experimental biological data from an experimental biological data set into a processor in a format to be displayed in matrix form, with the matrix containing rows pertaining to items upon which experiments were performed, and at least one column containing values obtained as a result of the experiments performed on the corresponding items; inputting supporting data from at least one supporting data set into the processor in a format to be displayed in the same matrix with the experimental biological data, wherein the supporting data corresponds to the items in the rows and provides at least one column of supporting data values; operating the processor to produce an image on a display, the image defining a two dimensional representation of the matrix in a compressed format, wherein the experimental values are expressed graphically in compressed format with the size and direction of the graphical representation indicating the relative value of the experimental values; and wherein adjacent, like values in the supporting data columns are represented by a graphical block, line or other graphical representation; sorting at least one column of the matrix to arrange the column in an order of ascending or descending values; and viewing the data to identify similarities or trends among the graphical representations of the data in any of the columns.

The data may be de-normalized prior to inputting it to form the single matrix, so the supporting data is repeated for each item from the experimental data that it relates to.

The experimental data may comprise microarray data, data from Taqman experiments, protein identification data from mass spectrometry or gel electrophoresis, or cell localization information from flow cytometry, or other types of biological data, for example.

The supporting data may include phenotype information from clinical data or knockout experiments, genotype information from association studies and DNA microarray experiments, patient identification information, etc.

Graphical representations of interest in the compressed matrix display may be selected by one or more rows, and expanded to a non-compressed format for closer visual scrutiny of the values contained in those rows.

One or more columns of compressed data may be removed from the matrix to focus on the remaining columns thought to be more relevant to identifying a relationship, trend or correlation among the diverse data sets.

Expanded data may be compared with at least one of the data sets from which the data in the row or rows of the expanded data was originally inputted. All or a portion of one or more data sets may be overlaid on the compressed matrix display for easier comparison of compressed or expanded data in the matrix with the information in the data set or data sets from which the compressed matrix was generated.

The present invention further includes overlaying a graphical representation of the data set, such as a heat map of an experimental data set, on the view displaying the data in compressed format.

Compressed or expanded data in the matrix may be highlighted, with corresponding automatic highlighting of data in the corresponding data sets from which the highlighted matrix data was originally inputted.

The present invention may further include a pop-up feature to compare data from the matrix with one or more of the originating data sets, or a switch screen function can be provided for switching between the matrix view and one or more of the originating data sets.

The present invention may place graphical representations of the values contained in the experimental data column of the expanded rows of the matrix. For example, the expanded experimental data values may be color coded in red and green hues, with green hues representing various levels of down-regulation and red hues representing various levels of up-regulation of the items, respectively.

The present invention may monitor the number of rows included in each block, line or other graphical representation formed to indicate locations of adjacent, like values in the supporting data columns, and overlay a descriptive label over each block, line or graphical representation which includes at least a minimal predetermined number of rows, wherein the descriptive label describes a common feature of the data represented by the block, line or other graphical representation.

The present invention may further include performing at least one computational analysis on at least one column of values to determine values for a new column to be added to the matrix, and displaying the determined values in the new column in the matrix.

A system for visually inspecting diverse, very large data sets of biological data to identify trends, correlations or relations among data from the data sets is provided to include means for de-normalizing experimental data contained in an experimental biological data set and supporting data contained in at least one biological supporting data set; means for inputting the de-normalized experimental biological data and the de-normalized biological supporting data to a processor; means for controlling the processor to generate a matrix containing all of the de-normalized data inputted from the experimental biological data set and each supporting data set, wherein the matrix contains rows pertaining to items upon which experiments were performed, at least one column containing values obtained as a result of the experiments performed on the corresponding items, and at least one column containing supporting data corresponding to the items in the rows;

means for displaying the matrix, in compressed format, on a display screen such that all of the data is graphically represented on the display screen, wherein the experimental values are expressed graphically in compressed format with the size and direction of the graphical representation indicating the relative value of the experimental values; and wherein adjacent, like values in the supporting data columns are represented by a block, line or other graphical representation; means for sorting any selected column of the matrix to arrange the column in an order of ascending or descending values; and means for expanding one or more selected rows of the matrix to be displayed in a non-compressed format.

The system may further include means for overlaying a graphical representation of the experimental data set on the display of the matrix.

The system may further include means for substantially simultaneously highlighting data in the matrix and data in at least one of the data sets from which the data was inputted to generate the matrix.

Means for displaying graphical representations of the experimental data displayed in expanded form may be provided, where the graphical representations correspond to graphical representations of the experimental or supporting data from which the matrix data originated.

The system may further include means for monitoring the number of rows included in a block, line or other graphical representation formed to indicate locations of adjacent, like values in the supporting data columns, and means for overlaying a descriptive label over each block, line or other graphical representation which includes at least a minimal predetermined number of rows. The descriptive labels describe a common feature of the data represented by the block, line or other graphical representation.

The system may include means for performing at least one computational analysis on at least one column of values of the matrix to determine values for a new column to be added to the matrix, and displaying the determined values in the new column in the matrix. The system may perform clustering, classification, statistical analysis, error modeling or other computations on the data already loaded into the matrix.

A computer-readable medium carrying one or more sequences of instructions from a user of a computer system for visually inspecting diverse, very large data sets of biological data to identify trends, correlations or relations among data from the data sets is provided, wherein the execution of the one or more sequences of instructions by one or more processors cause the one or more processors to perform the steps of: de-normalizing experimental data contained in an experimental biological data set and supporting data contained in at least one biological supporting data set; inputting the de-normalized experimental biological data and the de-normalized biological supporting data to the one or more processors; controlling the processor to generate a matrix containing all of the de-normalized data inputted from the experimental biological data set and each supporting data set, wherein the matrix contains rows pertaining to items upon which experiments were performed, at least one column containing values obtained as a result of the experiments performed on the corresponding items, and at least one column containing supporting data corresponding to the items in the rows; displaying the matrix, in compressed format, on a display screen such that all of the data is graphically represented on the display screen, wherein the experimental values are expressed graphically in compressed format with the size and direction of the graphical representation indicating the relative value of the experimental values; and wherein adjacent, like values in the supporting data columns are represented by a block, line or other graphical representation; sorting any selected column of the matrix to arrange the column in an order of ascending or descending values; and expanding one or more selected rows of the matrix to be displayed in a non-compressed format.

The medium may further include instructions for the performance of the steps of: overlaying a graphical representation of the experimental data set on the display of the matrix; substantially simultaneously highlighting data in the matrix and data in at least one of the data sets from which the data was inputted to generate the matrix; displaying graphical representations of the experimental data displayed in expanded form, which correspond to graphical representations of the experimental data which are contained in the experimental data set; monitoring the number of rows included in each block, line or other graphical representation formed to indicate locations of adjacent, like values in the supporting data columns and overlaying a descriptive label over each block, line or other graphical representation which includes at least a minimal predetermined number of rows, wherein the descriptive label describes a common feature of the data represented by the block, line or other graphical representation; and/or performing at least one computational analysis on at least one column of values of the matrix to determine values for a new column to be added to the matrix, and displaying the determined values in the new column in the matrix.

These and other objects, advantages, and features of the invention will become apparent to those persons skilled in the art upon reading the details of the invention as more fully described below.

### BRIEF DESCRIPTION OF THE DRAWINGS

FIG. 1 shows a screen shot, according to the present invention, of a view of 30 DNA gene expression microarrays expressed graphically on a table image along with clinical data and patient cluster data relating to the patients whose DNA the microarray experiments were conducted.

FIG. 2 shows a screen shot of the data in FIG. 1 having been sorted by patient cluster and invasive ability.

FIG. 3 shows a screen shot of a subset of the data shown in FIG. 2, with less informative columns of data having been removed.

FIG. 4 shows the same arrangement of data as shown in FIG. 3, after having zoomed in on patients 52 and 54.

FIG. 5 shows the same arrangement of data as shown in FIG. 3, wherein additionally, expression ratios have been color-coded, in proportion to degrees of up-regulation and down-regulation. Additionally, FIG. 5 shows labeling of block data.

FIG. 6 shows color-coding of expanded log ratio data values, wherein the color-coding also graphically shows the position of the value as shown in the compressed data.

FIG. 7 shows color-coding of the expanded log ratio data wherein the color-coding corresponds exactly to the heat map color-coding from which the data was derived.

FIG. 8 shows the negative log values folded over so as to extend to the right along with the positive log values.

FIG. 9 shows a version of the data set where microarray data is expressed as simple ratios vs. log ratios.

FIG. 10 shows a dialog with entries for computing a column of log ratio data from simple ratio data.

FIG. 11 shows the additional computed column of log ratio data added to the compressed matrix display.

FIG. 12 shows a dialog with entries for computing a k-Means clustering of the log ratio data.

FIG. 13 shows the additional computed column of cluster designations added to the compressed matrix display.

### DETAILED DESCRIPTION OF THE INVENTION

Before the present methods, tools and system are described, it is to be understood that this invention is not limited to particular data sets, manipulations, tools or steps described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and articles of manufacture similar or equivalent to those described herein can be used in the practice or testing of the present invention, the preferred methods and articles are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “a data set” includes a plurality of such data sets and reference to “the step” includes reference to one or more such steps and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### DEFINITIONS

The term “cell”, when used in the context describing a data table, refers to the data value at the intersection of a row and column in a spreadsheet-like data structure; typically a property/value pair for an entity in the spreadsheet, e.g. the expression level for a gene.

“Color coding” refers to a software technique which maps a numerical or categorical value to a color value, for example representing high levels of gene expression as a reddish color and low levels of gene expression as greenish colors, with varying shade/intensities of these colors representing varying degrees of expression.

The term “data mining” refers to a computational process of extracting higher-level knowledge from patterns of data in a database. Data mining is also sometimes referred to as “knowledge discovery”.

The term “de-normalize” refers to the opposite of normalization as used in designing database schemas. Normally, when designing efficiently stored relational data, one attempts to reduce redundant entries by creating tables containing single instances of data whenever possible. Fields within these tables point to entries in other tables to establish one to one, one to many or many to many relationships between the data. De-normalizing means to flatten out this space efficient relational structure, often for the purposes of high speed access that avoid having to follow the relationship links between tables.

The term “down-regulation” is used in the context of gene expression, and refers to a decrease in the amount of messenger RNA (mRNA) formed by expression of a gene, with respect to a control.

“Gel electrophoresis” refers to a biological technique for separating and measuring amounts of protein fragments in a sample. Migration of a protein fragment across a gel is proportional to its mass and charge. Different fragments of proteins, prepared with stains, will accumulate on different segments of the gel. Relative abundance of the protein fragment is proportional to the intensity of the stain at its location on the gel.

The term “gene” refers to a unit of hereditary information, which is a portion of DNA containing information required to determine a protein's amino acid sequence.

“Gene expression” refers to the level to which a gene is transcribed to form messenger RNA molecules, prior to protein synthesis.

“Gene expression ratio” is a relative measurement of gene expression, wherein the expression level of a test sample is compared to the expression level of a reference sample.

A “gene product” is a biological entity that can be formed from a gene, e.g. a messenger RNA or a protein.

A “heat map” is a visual representation of a tabular data structure of gene expression values, wherein color-codings are used for displaying numerical values. The numerical value for each cell in the data table is encoded into a color for the cell. Color encodings run on a continuum from one color through another, e.g. green to red or yellow to blue for gene expression values. The resultant color matrix of all rows and columns in the data set forms the color map, often referred to as a “heat map” by way of analogy to modeling of thermodynamic data.

A “hypothesis” refers to a provisional theory or assumption set forth to explain some class of phenomenon.

An “item” refers to a data structure that represents a biological entity or other entity. An item is the basic “atomic” unit of information in the software system.

The term “mass spectrometry” refers to a set of techniques for measuring the mass and charge of materials such as protein fragments, for example, such as by gathering data on trajectories of the materials/fragments through a measurement chamber. Mass spectrometry is particularly useful for measuring the composition (and/or relative abundance) of proteins and peptides in a sample.

A “microarray” or “DNA microarray” is a high-throughput hybridization technology that allows biologists to probe the activities of thousands of genes under diverse experimental conditions. Microarrays function by selective binding (hybridization) of probe DNA sequences on a microarray chip to fluorescently-tagged messenger RNA fragments from a biological sample. The amount of fluorescence detected at a probe position can be an indicator of the relative expression of the gene bound by that probe.

The term “promote” refers to an increase of the effects of a biological agent or a biological process.

A “protein” is a large polymer having one or more sequences of amino acid subunits joined by peptide bonds.

The term “protein abundance” refers to a measure of the amount of protein in a sample; often done as a relative abundance measure vs. a reference sample.

“Protein/DNA interaction” refers to a biological process wherein a protein regulates the expression of a gene, commonly by binding to promoter or inhibitor regions.

“Protein/Protein interaction” refers to a biological process whereby two or more proteins bind together and form complexes.

A “sequence” refers to an ordered set of amino acids forming the backbone of a protein or of the nucleic acids forming the backbone of a gene.

The term “overlay” or “data overlay” refers to a user interface technique for superimposing data from one view upon data in a different view; for example, overlaying gene expression ratios on top of a compressed matrix view.

A “spreadsheet” is an outsize ledger sheet simulated electronically by a computer software application; used frequently to represent tabular data structures.

The term “up-regulation”, when used to describe gene expression, refers to an increase in the amount of messenger RNA (mRNA) formed by expression of a gene, with respect to a control.

The term “UniGene” refers to an experimental database system which automatically partitions DNA sequences into a non-redundant sets of gene-oriented clusters. Each UniGene cluster contains sequences that represent a unique gene, as well as related information such as the tissue types in which the gene has been expressed and chromosome location.

The term “view” refers to a graphical presentation of a single visual perspective on a data set.

The term “visualization” or “information visualization” refers to an approach to exploratory data analysis that employs a variety of techniques which utilize human perception; techniques include graphical presentation of large amounts of data and facilities for interactively manipulating and exploring the data.

The present invention provides efficient methods of inspecting very large data sets of microarray, protein array or other large-scale biological experiments along with other relevant supporting data sets, in order to visually identify correlations, trends or similarities between the experimental data and supporting data or within the experimental data based on manipulating the supporting data. According to the present invention, widely diverse but related and potentially correlated data (such as gene expression and clinical observations) can be combined and studied. Further, by using an easily manipulated graphical rendering of the total data set (experimental data set combined with any supporting data sets) it is possible to easily search for meaningful correlations and trends using innate human pattern recognition. This allows powerful ad-hoc analysis of the data to be performed that is otherwise inaccessible to the researcher or scientist examining such data.

The present methods employ a visualization tool known as Table Lens, which allows the diverse data sets to be displayed and inspected simultaneously in graphical form on a single display. In particular, in the examples discussed, the system according to the present invention was based on a product known as Eureka, by Inxight. A complete description of the functionality of Table Lens can be found in U.S. Pat. Nos. 5,632,009; 5,880,742 and 6,085,202, each of which is incorporated herein, in its entirety, by reference thereto.

Using the present techniques and modifications, an extremely powerful tool and procedures for visualizing the massive data sets generated by high-throughput experiments such as DNA microarrays in combination with supporting data is provided. The results of these experiments, as well as the supporting data, can be visually manipulated to look for trends and correlations using simple human intelligence in lieu of more sophisticated analytical tools such as clustering or classification algorithms. Nothing precludes using these algorithmic tools, however, and the calculated data can even be incorporated into the data set being examined according to the present invention.

However, the human mind has adapted over evolution to have powerful pattern matching abilities, and the present invention leverages this ability to permit a high degree of ad-hoc high-level analysis and discovery to be performed. Algorithmic techniques are quite powerful, but usually directed toward looking at specific pre-defined correlations or trends. The present invention facilitates approaching the data with no particular predisposition and can be used to provide insight as to which computational techniques might be useful.

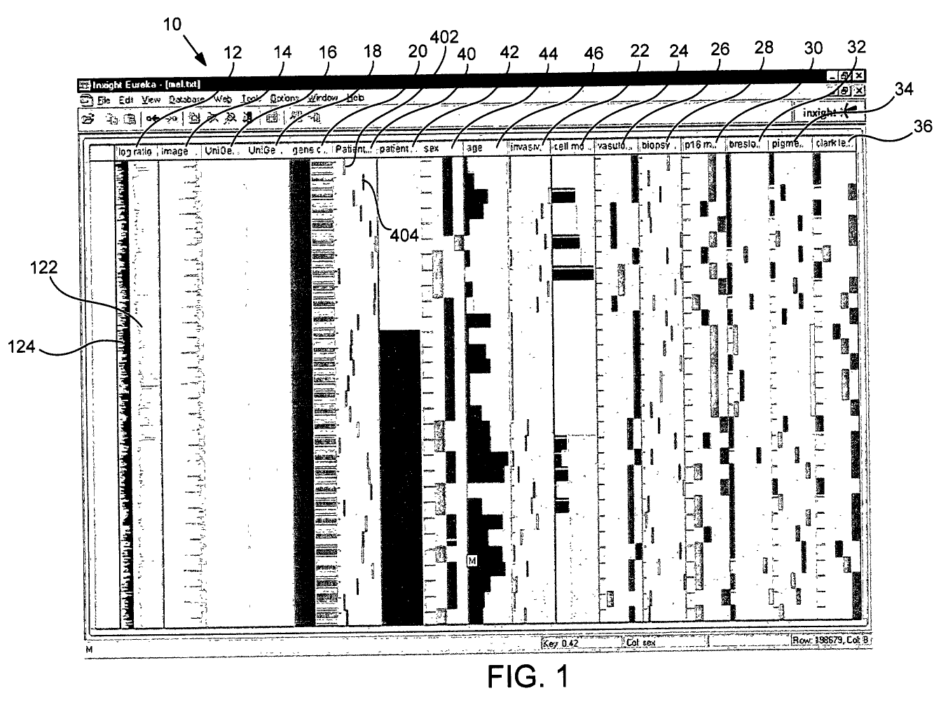

Turning now to FIG. 1, an example display is shown in which three diverse data sets have been loaded into the system for viewing and analysis with regard to possible trends, correlations, or other relationships of interest. In this case, a number of DNA microarray experimental results pertaining to melanoma were considered. The microarray experimental data was obtained from the National Human Genome Research institute of the National Institutes of Health. Further details regarding the microarray data can be found in Bittner et al., “Molecular classification of cutaneous malignant melanoma by gene expression profiling”, Nature, vol. 406, August, 2000, which is incorporated herein, in its entirety, by reference thereto.

Microarray experiments were performed on thirty one subcutaneous melanoma patients and seven patients without subcutaneous melanoma (controls). A considerable amount of clinical data was also generated which supplements the microarray data. The clinical data characterized the type and severity of the various melanomas. While the clinical data showed little correlation, using computational techniques (discussed in the article by Bittner et al., cited above), a set of informative genes were discovered which indicated a patient cluster of related melanomas. Based on the known function of the informative genes further data was collected which did in fact correlate with the gene-predicted properties of this cluster.

The microarray data, supporting clinical data, and patient identification data were all loaded into the system so as to display all of the data on a single screen in a compressed format. Data can be loaded into this format by a number of techniques including: inputting tab-delimited ASCII text; importing from an Excel spreadsheet, or importing results of an SQL query from a database. In the following example, the data from each of the three diverse data sets was assembled in an Excel spreadsheet and then imported into the compressed matrix 10 shown in FIG. 1.

FIG. 1 shows a view 10 of thirty (of the thirty one melanoma patients) DNA gene expression microarrays. For each patient 8066 individual microarray measurements are displayed in the column 12 labeled “log ratio” (i.e. the standard log 10 ratio of the signal measurements made for each feature of the array). The table shown is thus a very dense graphical display representing 241,980 rows of data entirely visible on a single standard computer display. The column 14 labeled “image” contains the cloneID for the CDNA having been deposited on the microarray with respect to each individual microarray reading identified in column 12. Column 16 (“Unigene”) contains the Unigene Cluster ID that further identifies the CDNA having been deposited on the microarray.

The “Unigene Description” (column 18) gives the name of each unigene cluster identified, respectively. The “gene cluster” column 20 marks those genes that were determined to be particularly informative (as noted above). Genes thought to be particularly informative are indicated with a value of one, while all other genes are assigned a value of zero. An example of one such gene in the set was WNT5A.

In addition to loading the microarray experimental data, clinical data relating to the microarray experiments were loaded into the same matrix view 10, and included invasive ability observed in the melanoma for that particular sample (column 22), cell mobility 24, vasculogenic mimicry (the relative ability of the sample to form tubular networks that resemble embryonic vasculogenesis) 26, biopsy site (the location on the patient's body from which the biopsy was taken) 28, P16 mutation (a particular gene mutation that the researchers were interested in as possibly being particularly relevant) 30, Breslow thickness 32, pigmentation 34 and Clark's level 36.

A third data set pertaining to patient identification was incorporated into the matrix to form columns 40, 42, 44 and 46 containing patient identification number, patient cluster (whether a particular patient belonged to an invasive or non-invasive classification, as determined clinically), sex of the patient and age of the patient respectively. The resulting table view 10 links that patient identifying information with the microarray experiments and the supporting clinical data. Thus, the rows of the matrix view each include the clinical data as well as patient cluster and identification of which genes are being measured on the microarray. However, the present invention is not limited to the incorporation of three data sets for visualization, as two diverse data sets, or more than three diverse data sets could be incorporated into a single matrix to visualize the data together in an effort to identify correlations, trends, similarities, outliers, etc.

The underlying table is constructed by de-normalizing (in the database sense) the gene and patient data. In this way, each row of the matrix includes the patient and clinical data which were generated for the particular gene that is shown in the microarray expression data. By de-normalizing the data, the data from the patient information data set and the clinical data set (i.e., data in columns 22-36 and 40-46) is repeated for each gene measured by that patient's microarray. For normal tabular data this is largely uninformative to construct such a table, but for the present invention, this technique leads to some potentially useful pattern recognition techniques.

The underlying software greatly compresses the data so as to be able to contain it, and view it in condensed form, on a single screen. Because the visualization is highly compressed, graphical values are displayed to represent the compressed data. The graphical values displayed in FIG. 1 show the log ratios 12 of the microarray experiments displayed as horizontal lines, with white lines 122 indicating the maximum values in the displayable areas and the dark (actually blue, although this is not distinguishable in FIG. 1) lines 124 indicating the minimum values. This is particularly useful in the log ratio column 12 as there are actually many values represented within a particular “pixel” row due to the high compression of the data to fit within this display.

A second important feature is that blocks of adjacent similar data will appear as colored rectangles. Since some data can be designated as “categories” vs. numerical measurements, this is quite useful. In the display of FIG. 1, it can be appreciated that the patient id column 40 clearly shows blocks 402, 404, etc. of rows corresponding to each patient. Additionally, the data contained in the view 10 can be selectively sorted. Depending upon how the data is sorted, new blocks of adjacent similar data may appear, thereby indicating, from a macro or general view, a similarity between the adjacent data. However, in the arrangement as shown in FIG. 1, it is difficult to identify any really relevant correlations as no really meaningful sort order has been chosen yet.

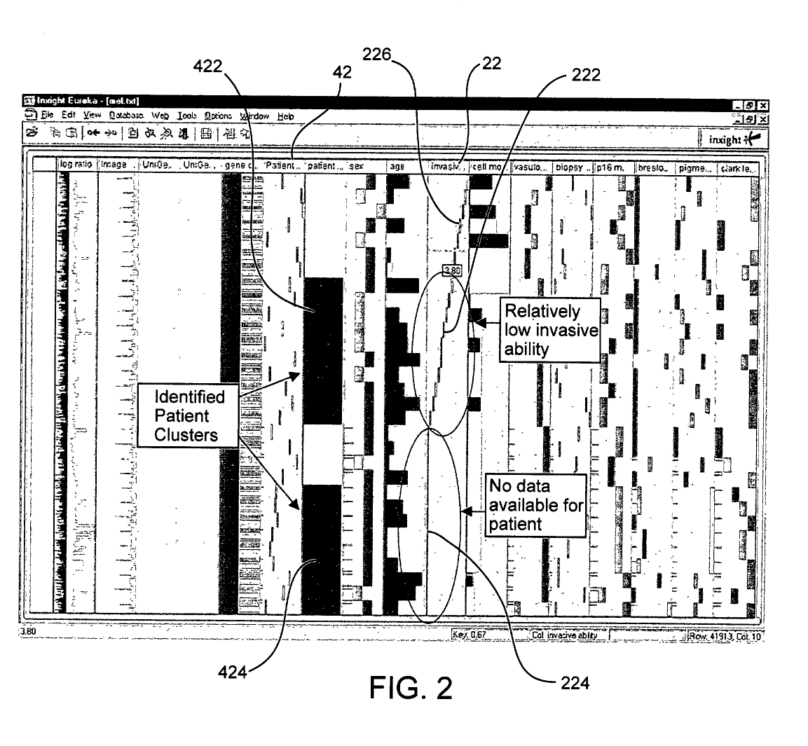

Turning to FIG. 2, a view of the data sets is shown after having sorted the data first by patient cluster 42, and then by invasive ability 22. As clearly shown, the patient cluster sorting generated patient cluster blocks 422 and 424. This procedure was carried out in an effort to verify the assertion made in the Bittner et. al. article (identified above) that the patient cluster assignment that was made in that study, based on informative genes that were identified in the study does indeed correspond to low invasive ability of the malignancy. As a result of the second sorting according to invasive ability, a clear relationship can be seen among the patient cluster 422 and the invasive ability values 222 which are clearly lower than the invasive ability values 226 corresponding to those patients not belonging to patient cluster 422 and which are shown above patient cluster 422 in FIG. 2. The invasive values 224 corresponding to patient cluster 424 appear as a straight vertical line because a measurement of invasive ability was not made in regard to this group of patients. Therefore, what might appear at first glance as a disparity is still consistent with the assertion being examined.

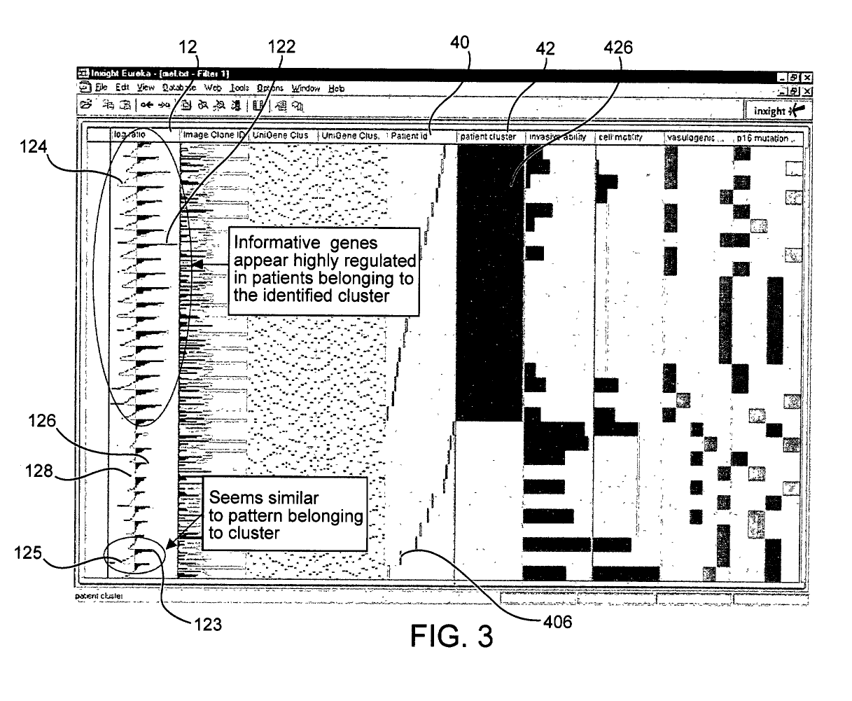

Further manipulation of the data sets was carried out to obtain a more striking insight. Initially, columns of data which were identified by the Bittner et al. article as being not “specifically associated” with the identified patient cluster group were removed as being considered “not informative”. Specifically, the columns of data containing sex 44, age 46, Breslow thickness 32, pigmentation 34 and Clark's level 36 were removed. The data was further filtered to remove rows of data that did not include those genes which were identified in the Bittner et al. article, using computational techniques, as being informative to determining the patient cluster assignment. FIG. 3 shows the resultant data set, after removing the columns and rows as described, and then sorting the reduced data set by log ratio 12, then patent id 40 and finally by patient cluster 42.

When using the present system, the user must be mindful of the sort order by which the data has been sorted. For example, in FIG. 3, care should be taken not to misinterpret the log ratio data 12, as this data, in FIG. 3, has been sorted by highest to lowest regulation for each patient, since the data was sorted by patient id 40 subsequent to sorting by log ratio 10. Consequently, not all patients are displaying ratio in precisely the same order, although this sort profile does give a overall impression of the distribution of regulation within the set of genes for each patient.

Further, due to the sorting by patient cluster 42 following sorting by log ratio 10 and patient id 40, this sort arrangement does clearly show that those patients belonging to the “informative cluster” 426 show a wide distribution of relatively high 122 and low 124 gene regulation, while generally those patients not in the cluster do not exhibit extremely high or low expression of these genes, for example, see the peaks 126 and 128, which are not nearly as extreme. However, by the simple manipulations performed to generate this display there are a few inconsistencies among the patients not categorized as part of the cluster 426. For example, the peaks 123 and 125 clearly show a distribution much more characteristic of the patients belonging to the cluster 426, although the previous computational techniques performed on the data did not identify this patient as belonging to the informative cluster 426.

The graphically represented peaks 123 and 125, and the graphical representation (vertical line) 406 representing the patient id are not directly informative to the user in identifying the log ratios and the corresponding patient information of the patient that potentially belongs to the informative cluster 426, since the data is compressed, as described above, and therefore contains many values per pixel, which are not perceptible by the human eye. The individual patient information and corresponding log ratio values can be visualized by zooming in on the area of interest, which is accomplished by clicking or dragging over the area of interest, as described in Inxight Eureka Version 1.2 Tutorial© 1999-2001, Inxight Software, Inc., which is incorporated herein, in its entirety, by reference thereto.

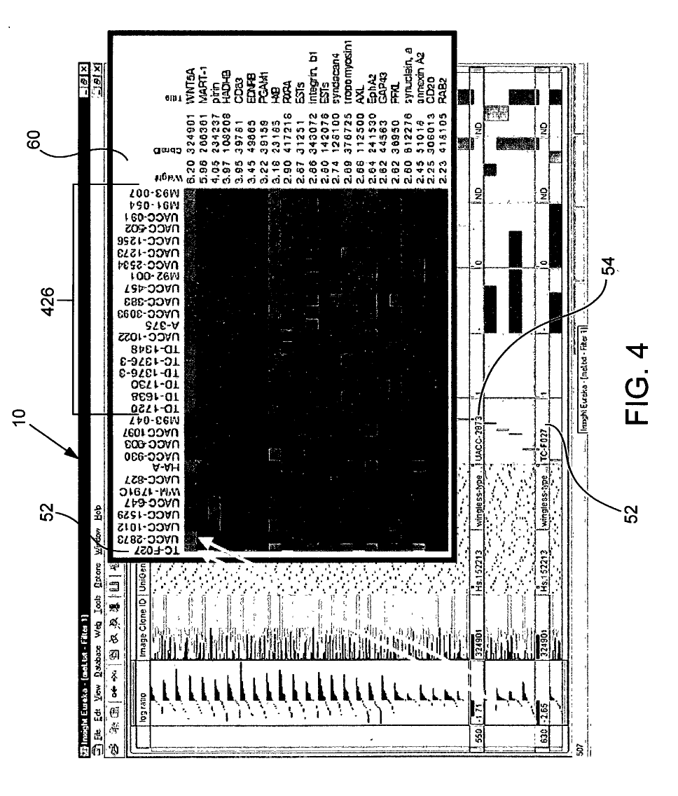

FIG. 4 shows the same arrangement of data as shown in FIG. 3, after having zoomed in on patients 52 and 54 (patients TC-F027 and UACC-2873, respectively). It is noted that the anomalous patient 52 was discovered while testing the concepts presented in the present invention. The present invention further includes features for linking a heat map (as shown in FIG. 4) or other tabular or graphical representation to the data which is displayed by the main display 10. With this feature, the heat map can be selected generally by switching screens to the heat map 60 or other corresponding tabular or graphical display which corresponds to the data being viewed in 10, for a contextual view. Alternatively, the user may choose the pop up heat map when in the zoom mode and examining a single or small group of data. By using the spotlight feature of the Table Lens, the pop-up heat map or other corresponding image also highlights the particular data elements of interest, which can then be readily referenced by the user.

The overlaid heat map 60 shown in FIG. 4, is the cluster diagram heat map from the Bittner et al. paper identified above, from which the log ratio data was taken for the display 10. By popping up, overlaying, or switching screens to view the heat map 60, the user can view the cluster diagram showing the usual red/green “heat map” visualization. By viewing the pattern of the variations in red/green hues of the patients identified in cluster 426, and comparing the corresponding red/green hues of expression levels of patient 52, it can be seen that patient 52 clearly shows a generally matching pattern, corroborating that the pattern identified in the above described sorting procedure does in fact exist in the original analysis. Even though FIG. 4 does not show the red/green hues which would be readily discernable when using the invention as described, even the gray-shade representation in FIG. 4 indicates that the pattern shown for TC-F027 in the heat map 60 does look more similar to the clustered patients 426 than the non-clustered ones. A more recent paper by Heydebreck et. al., “Identifying Splits with Clear Separation: A New Class Discovery Method for Gene Expression Data”, Bioinformatics 1:1-8 (2001), which is incorporated herein, in its entirety, by reference thereto, uses a different algorithm to cluster the melanoma data, and further corroborates the finding noted above, indicating that TC-F027 was probably misclassified in the original Bittner et al. publication.

Thus, the methods described above indicate an alternative approach to identifying relationships that exist among large data sets of diverse biological information, in a way that can be visually and directly observed by the user. While these observations and results may be obtained by other means, such as the computational methods in Heydebreck et. al. article with regard to the example described above, the present inventions provides a relatively simple and direct visualization technique which can be used to obtain independent results of correlations, relationships, and similarities among diverse biological data sets, as well as to corroborate results obtained by other current analysis techniques. Further, the present invention may be useful in supplementing previously conducted analysis techniques, or to correct results which have not been interpreted entirely correctly.

Thus, a totally independent result can be found, as in the case of the discovery of the anomaly of patient TC-F027, which was discovered using the techniques as discussed above. It wasn't until this discovery was made by the present inventors, that a search was made for verification which was found in the Heydebreck et al. article. The results derived by computational means were validated by independent interactive visualization according to the present invention. In the above example, the Bittner et al. patient cluster was validated and supplemented by the addition of patient TC-F027, and the Heydebreck et. al. computational results were validated.

It is noted that the present methods may be applied, not only to data which has been previously analyzed by other techniques to provide groupings of data to begin with, but that the present invention can be used similarly as an initial approach to analyzing experimental data together with one or more sets of clinical or other supporting data to investigate trends, correlations, or other relationships among the data sets which could form a starting point indicating which data form the data sets is relevant to examine more closely, and possibly which data should be examined by more traditional computational approaches. By leveraging human pattern recognition early in the process, more informed and targeted computational methods can be applied.

The present invention is not intended to replace computational approaches to discovering trends, correlations and relationships among biological data, but rather is intended to complement these other forms of discovery. Analysis by the present methods can lead to an independent and immediate result as in the case described above, can lead to a more informed computational stage, and/ or can incorporate computations as additional supplemental data in the analysis techniques of the present invention.

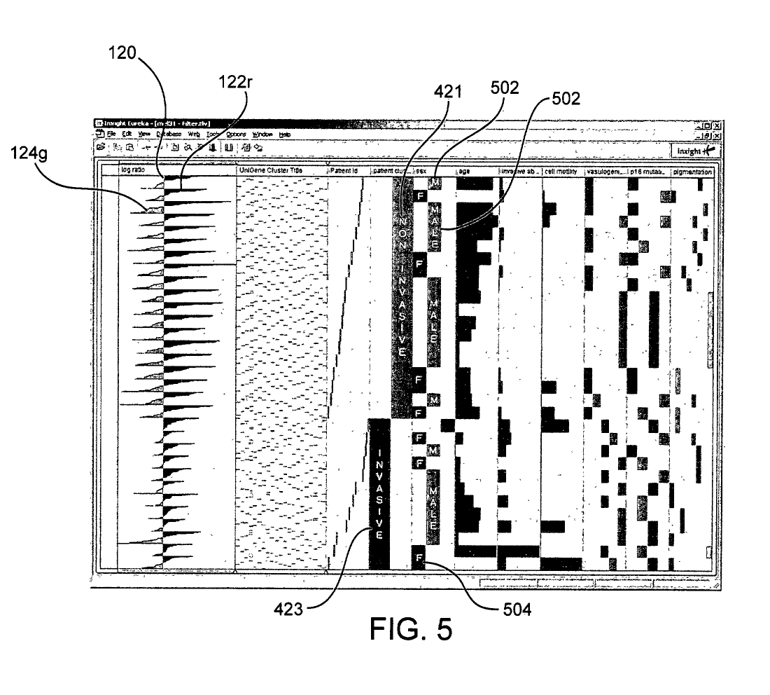

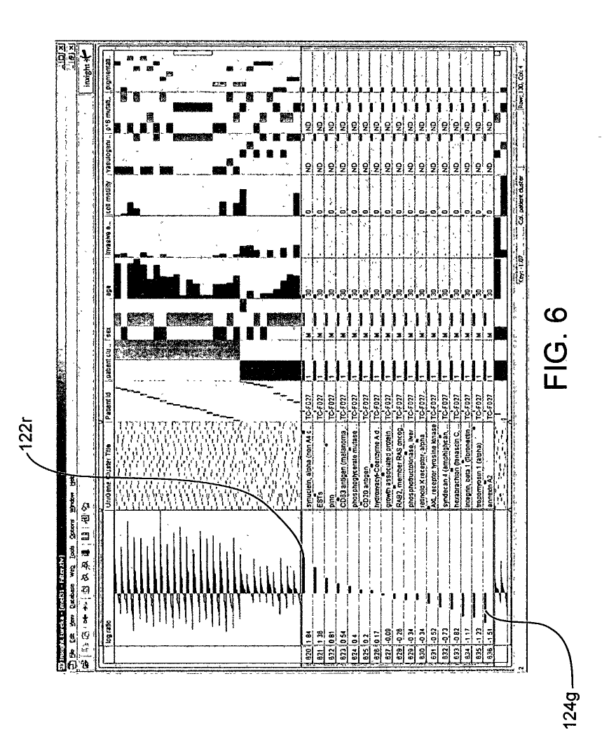

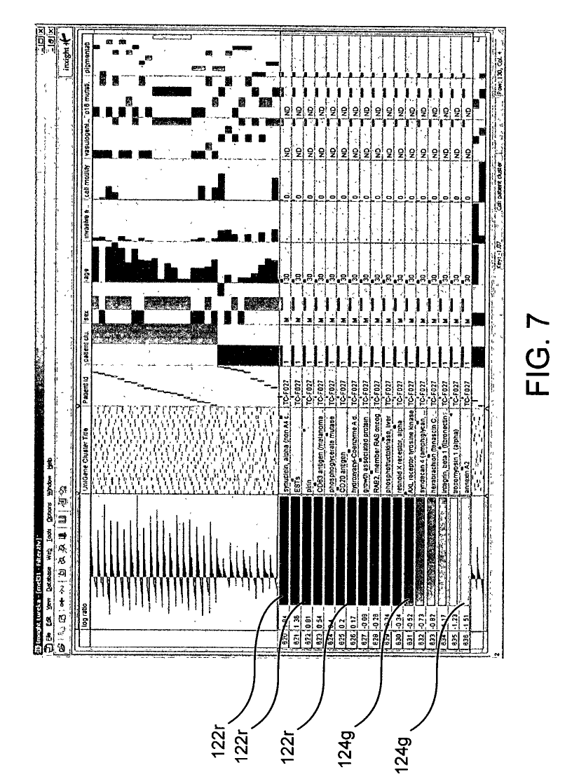

In addition to the usual graphical display shown in the above examples, the present invention may provide red/green intensity color-coding to the log ratio displays, to give the system a better intuitive feel to biological researchers, who are already conditioned to the red and green hues presented in heat maps which graphically present log ratios from microarray experiments. As such, gene or protein expression ratios may be color-coded, as shown in FIG. 5, to show red 122r in proportion to up-regulation and green 124g in proportion to down-regulation, analogous to the coloring in the heat map 60 shown in FIG. 4 (although not shown in color). Further analogously to the heat map shown in FIG. 4, the color-coding of the gene expression ratios in FIG. 5 can be colored to vary in intensity from neutral 120 (which shows up as black on a heat map) to more intensely green as the distance increases to the left from neutral. Similarly, the intensity of the red color-coding 122r increases as the distance to the right from neutral 120 increases. The compressed display in this variation applies the red-green color-coding 122r, 124g to the line graphs as shown in FIG. 5. However, for the expanded data (e.g., rows 620-636 in FIG. 6), the standard heat map displays are inserted next to the log values. FIG. 7 shows the expanded data with color-coded representation that corresponds exactly to heat map color-coding. Thus, for example, data line 620 which has a higher log value that data line 622 has a color-coding bar 122r that is more intensely red than the color-coding bar 122r adjacent line 622. The log value for data line 626 is not much above neutral and, accordingly, the color bar 122r associated with line 626 is a very dark or dull red almost approaching the color black. Similarly, the color bar adjacent line 636 is a much more intense and brighter green that that adjacent line 631.

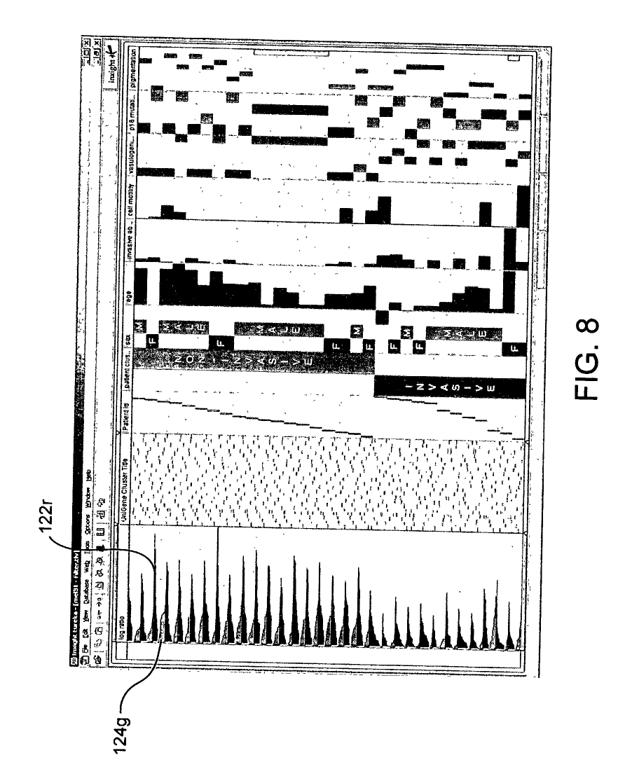

An additional feature that can be provided with the color-coded graphical representation of log values, is that the negative log values (i.e., green-encoded graphical markings) can be folded over so as to extend to the right along with the positive log values (i.e., the red-encoded graphical markings) as shown in FIG. 8. Because the red and green graphical representations can be easily visually distinguished, this feature can be useful for maximizing the resolution of the features presented on the screen, by allowing an effectively greater width on which to display the columns, while not significantly detracting from the readability of the log ratio data.

As noted above, when adjacent rows of compressed data have similar categories or the same value, the graphical display shows up as vertical lines and/or rectangles. The vertical lines and rectangles, by themselves, do not convey very much information to the user, other than alerting the user to the fact that a group of the same or similar values are arranged in that view. Also, the lines and rectangles leave a large blank (or colored) area on the display. The present invention monitors when such graphical representations are created and, when the number of rows exceeds a number calculated to be sufficiently large to permit the application of a readable, informative text label, data value, common value of the underlying block of similar values, or other group identifier, then such label, data value, common value of the underlying block of similar values, or other group identifier is generated and superimposed over the graphical representation in the case of a rectangle or the like, or may be superimposed or imposed adjacent a line representation, to further identify and describe the like data that is represented by that graphical representation. For example, FIG. 5 shows a patient cluster that has been labeled “Non-Invasive” 421 as well as one that has been labeled “Invasive” 423. Similarly, blocks indicating male and female patients have been labeled “Male” 502 (or simply “M” when the block is not large enough to fit the entire word “Male”) and F 504 (if the block was sufficiently larger, the entire label “Female” would appear). Since these labels apply to categories and are already in the system (and may even appear as tool tips) all that is required is to calculate the length of a block or rectangle to determine whether it large enough to display the rendered string (i.e., the label), or a suitable subset, such as an abbreviation, on it. Thus, a more informative sub-visualization of the graphical representation is provided as an adjunct to the overall display 10 and in particular to any graphical representation within the display 10 that meets the criteria for labeling. This labeling occurs as a natural course of using the present invention, and doesn't require any specific set up by the user. Further, the labels automatically appear and disappear with the forming and disbanding of the groupings that they represent, based upon the current sorting order of the data. In this way, the labels or other identifiers don't restrict the order in which the user can view the data, as any sort order can be applied in any order.

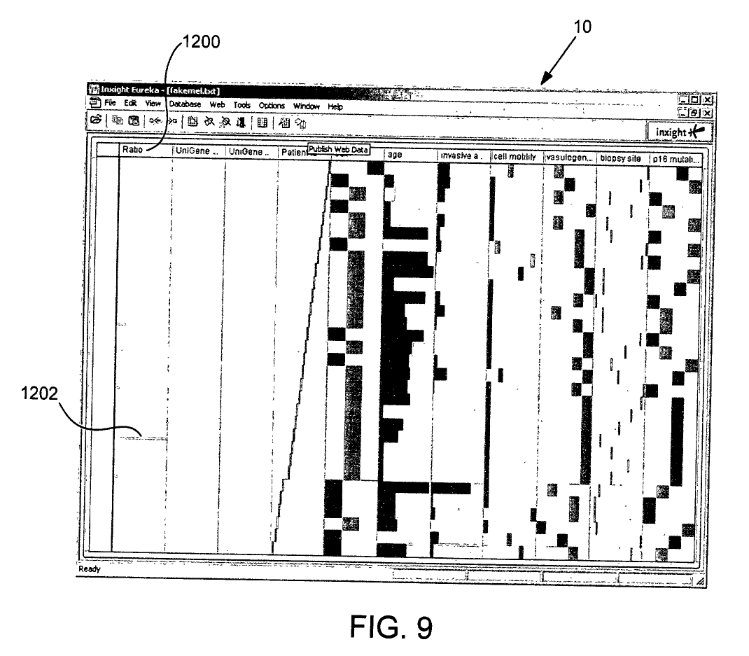

The present invention further presents the capability of performing computational analyses on the columns of data included within the data sets loaded. In addition to the ability to facilitate visual comparisons of large diverse data sets, as described above, computational techniques such as clustering or classification may be performed directly within the compressed data set provided, thus enabling immediate graphical feedback to aid in interpreting or validating the results. For example, FIG. 9 shows an example display in which the same three diverse data sets, referred to above with regard to FIG. 1, have been loaded into the system for viewing and analysis, but in this example, the gene expression data was reported using standard ratios 1200 (which may be indicated using red coloration for up-regulation values and green coloration for down-regulation values, for example). In this configuration, it is very difficult to spot any trends (or even very many values) in the “ratios” column 1200 because it is dominated by a very few ratios having extraordinarily high values, such as ratio 1202, for example. Because of the limited bandwidth of the “ratios” column, the few high ratio values make it very difficult, if not impossible to view the majority of the ratios values

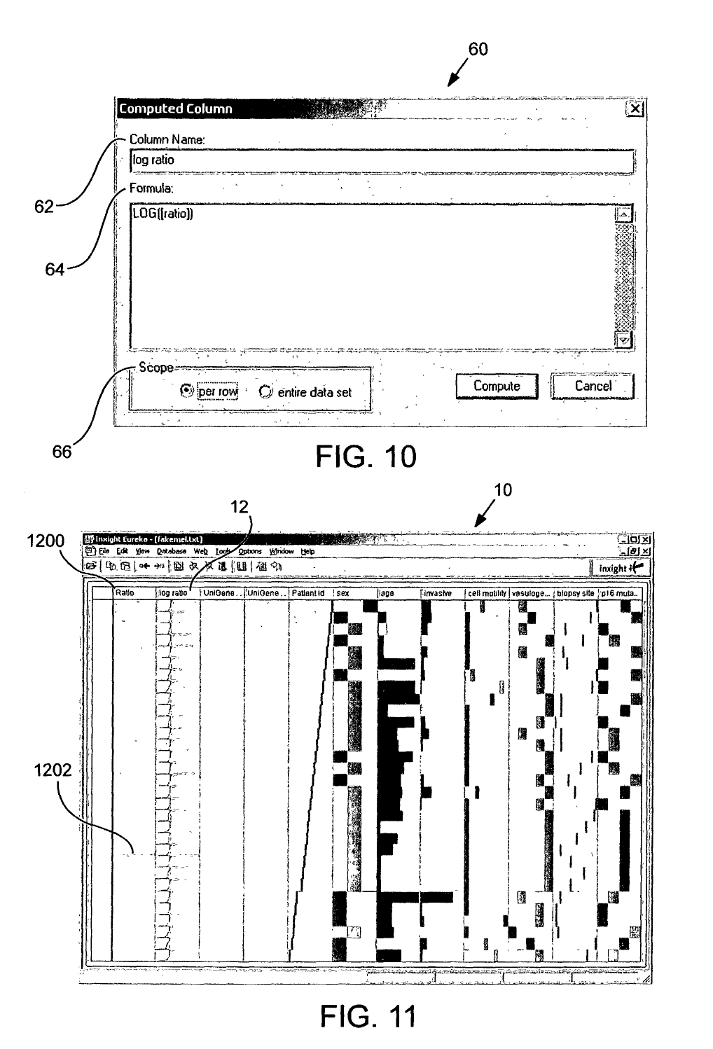

When faced with this situation, the information could possibly be presented in a more useful format by displaying the expression values in a “log-ratio” format, where log values of the expression values are displayed. To accomplish this manipulation, a menu item is invoked to bring up a “Computed Column” tool 60 as shown in the window view of FIG. 10. The computed column feature is used in this example to define a new column 62 called “log ratio”, and then a formula 64 is entered to compute the data to be entered into the new column 62 from data already loaded into the system (in this case, from the ratio data 1200). The scope of the computation in this example, is selected as a “per row” computation so that each value of the ratio column 1200 is individually entered into the formula 64 to compute respective log values that are used to populate the new column 12, as shown in FIG. 11. The log ratio results 12 in this example, are much more informative for visual trend spotting as they can be visually compared much more easily, as can be seen in FIG. 11.

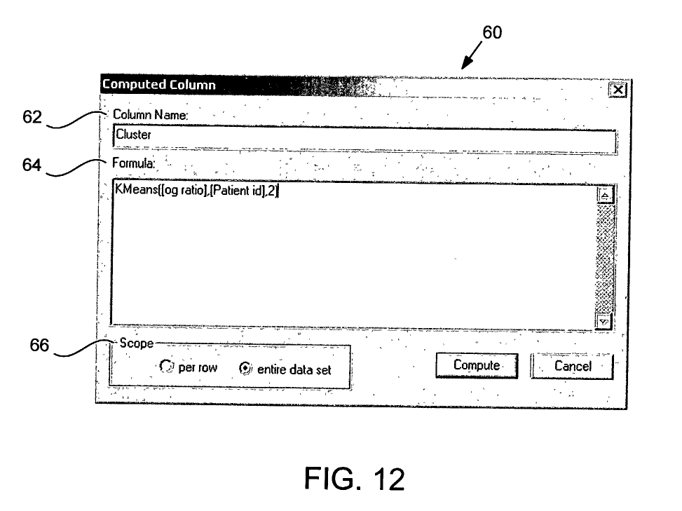

The information in the matrix 10 can be data mined by further manipulation, such as by performing clustering or categorization of data according to user defined parameters. For example, FIG. 12 shows a situation where the user has again invoked the computed column tool 60 to perform a clustering of the data according to two classes of melanoma data that are known to exist in the loaded data set. A new column titled “Cluster” is defined in the column name space 62, and then a formula 64 is entered to compute the data to be entered into the new column 62 from data already loaded into the system. Since the data is known to have two classes of patients (invasive and non-invasive melanomas), there was a likelihood that it could be informative and useful to perform k-Means clustering. This is a well known nonhierarchical method which divides the population of data into the required number “k” of clusters. In this case, a two cluster “K-means” was computed by selecting a predefined formula “KMeans” and specifying that the source data upon which to perform the calculations comes from the “log ratio” column, but that the data should be processed in an order determined by “patient id” values.

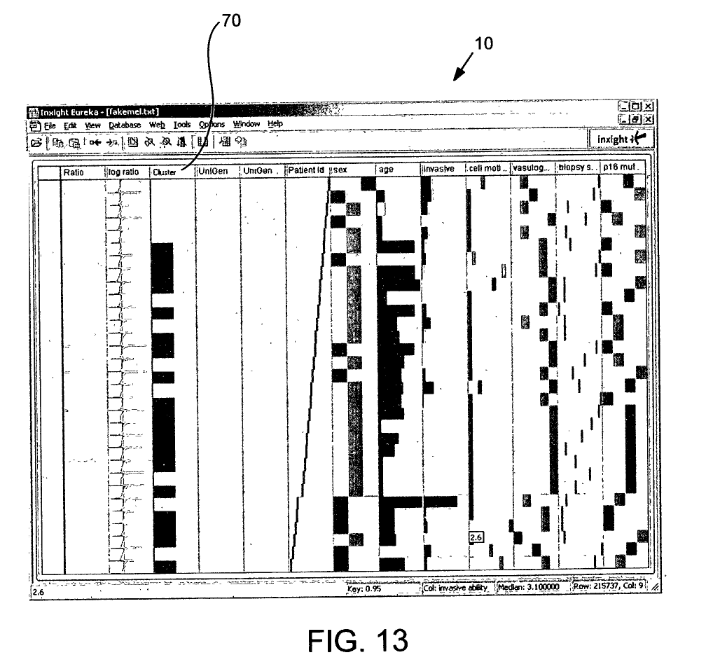

According to these instructions, the Kmeans algorithm de-normalizes the log ratio data according to patient id so that each feature vector processed by the clustering algorithm will be all the ratios for a given patient id. The numeral “2” in the entered formula 64 indicates that the results are desired to be formed into two clusters, which should correspond to the “invasive” and “non-invasive” melanoma data. The results of the clustering computation are shown in column 70 of FIG. 13. Although the above examples illustrated computational techniques for converting expression ratios to expression log ratios, and for clustering data according to patient id, the present invention is not limited to these two techniques. Various built-in algorithms for performing a variety of different types of calculations and manipulations of the compressed data can be included in the system, and/or the system can be set up to allow for plug-ins for various built in algorithms. Other types of calculations and manipulations that can be performed include clustering according to other user-defined parameters, classification, statistical analysis, error modeling, and the like.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, view, process, process step or steps, to the objective, spirit and scope of the present invention. All such modifications are intended to be within the scope of the claims appended hereto.

That which is claimed is:

1. A method of visually inspecting diverse, very large data sets of biological data to identify trends, correlations or relations among data from the data sets, said method comprising:
inputting experimental biological data from an experimental biological data set into a processor in a format to be displayed in matrix form, with the matrix containing rows pertaining to items upon which experiments were performed, and at least one column containing values obtained as a result of the experiments performed on the corresponding items;
inputting supporting data from at least one supporting data set into the processor in a format to be displayed in the same matrix with the experimental biological data, wherein the supporting data corresponds to the items in the rows and provides at least one column of supporting data values;
operating the processor to produce an image on a display, the image defining a two dimensional representation of the matrix in a compressed format, wherein the experimental values are expressed graphically in compressed format with the size and direction of the graphical representation indicating the relative value of the experimental values; and wherein adjacent, like values in the supporting data columns are represented by a graphical block, line or other graphical representation;
sorting at least one column of the matrix to arrange the column in an order of ascending or descending values; and
viewing the data to identify similarities or trends among the graphical representations of the data in any of the columns.

2. The method of claim 1, further comprising de-normalizing the data prior to said inputting.

3. The method of claim 1, wherein the experimental data comprises microarray data

4. The method of claim 1, wherein the experimental data comprises gene expression data or protein expression data.

5. The method of claim 1, wherein the supporting data comprises clinical data.

6. The method of claim 1, wherein supporting data is further inputted from a second supporting data set comprising patient identification data that links the experimental data with the clinical data.

7. The method of claim 1, further comprising the step of selecting a graphical representation of at least one row of the matrix that has been determined to potentially contain data relating to a trend, correlation or relation to some of the remaining data, and expanding the at least one row to a non-compressed format to view the values contained in the at least one row.

8. The method of claim 1, further comprising removing one or more columns to focus on the remaining columns thought to be more relevant to identifying a relationship, trend or correlation among the diverse data sets.

9. The method of claim 7, further comprising comparing the expanded data with at least one of the data sets from which the data in the row or rows of the expanded data was originally inputted.

10. The method of claim 9, wherein the expanded data is compared with the experimental data set.

11. The method of claim 10, comprising overlaying a graphical representation of the experimental data set on the view displaying the data in compressed format.

12. The method of claim 9, further comprising highlighting the expanded data, wherein the highlighted data is also automatically highlighted in the corresponding data sets from which the expanded data was originally inputted.

13. The method of claim 12, further comprising operating the processor to pop up, overlay or switch screens to a data set from which an expanded value was originally inputted; and comparing the highlighted values in the data set to corroborate or oppose the potential relationship, trend or correlation.

14. The method of claim 7, wherein the values contained in the experimental data column of the expanded rows contain graphical representations of the experimental data which are contained in the experimental data set.

15. The method of claim 14, wherein the experimental data is microarray data from a heat map and the values contained in the experimental data column of the expanded rows are color coded in red and green hues, with green hues representing various levels of downregulation and red hues representing various levels of up-regulation of the items, respectively.

16. The method of claim 1, further comprising the steps of:
monitoring the number of rows included in each block, line or other graphical representation formed to indicate locations of adjacent, like values in the supporting data columns; and
overlaying a descriptive label over each block or line representation which includes at least a minimal predetermined number of rows, wherein the descriptive label describes a common feature of the data represented by the block, line or other graphical representation.

17. The method of claim 1, further comprising performing at least one computational technique on at least one column of values to determine values for a new column to be added to the matrix, and displaying the determined values in the new column in the matrix.

18. The method of claim 17, wherein the at least one computational technique determines a cluster or classification of related values.

19. The method of claim 17, wherein the at least one computational technique includes a statistical algorithm.

20. The method of claim 17, wherein the at least one computational technique performs error modeling.

21. A system for visually inspecting diverse, very large data sets of biological data to identify trends, correlations or relations among data from the data sets, said system comprising:
means for de-normalizing experimental data contained in an experimental biological data set and supporting data contained in at least one biological supporting data set;
means for inputting the de-normalized experimental biological data and the denormalized biological supporting data to a processor;
means for controlling the processor to generate a matrix containing all of the denormalized data inputted from the experimental biological data set and each supporting data set, wherein the matrix contains rows pertaining to items upon which experiments were performed, at least one column containing values obtained as a result of the experiments performed on the corresponding items, and at least one column containing supporting data corresponding to the items in the rows;
means for displaying the matrix, in compressed format, on a display screen such that all of the data is graphically represented on the display screen, wherein the experimental values are expressed graphically in compressed format with the size and direction of the graphical representation indicating the relative value of the experimental values; and wherein adjacent, like values in the supporting data columns are represented by a block, line or other graphical representation;
means for sorting any selected column of the matrix to arrange the column in an order of ascending or descending values; and
means for expanding one or more selected rows of the matrix to be displayed in a non-compressed format.

22. The system of claim 21, further comprising means for overlaying a graphical representation of the experimental data set on the display of the matrix.

23. The system of claim 22, further comprising means for substantially simultaneously highlighting data in the matrix and data in at least one of the data sets from which the data was inputted to generate the matrix.

24. The system of claim 21, further comprising means for displaying graphical representations of the experimental data displayed in expanded form, said graphical representations corresponding to graphical representations of the experimental data which are contained in the experimental data set.

25. The system of claim 24, wherein the experimental data is microarray data from a heat map and the graphical representations of the expanded experimental data values comprise red and green hues, with green hues representing various levels of down-regulation and red hues representing various levels of up-regulation of the items, respectively.

26. The system of claim 21, further comprising means for monitoring the number of rows included in each said block, line or other graphical representation formed to indicate locations of adjacent, like values in the supporting data columns; and
means for overlaying a descriptive label over each said block, line or other graphical representation which includes at least a minimal predetermined number of rows, wherein the descriptive label describes a common feature of the data represented by the block, line or other graphical representation.

27. The system of claim 21, further comprising means for performing at least one computational technique on at least one column of values of the matrix to determine values for a new column to be added to the matrix, and means for displaying the determined values in the new column in the matrix.

28. The system of claim 27, wherein the at least one computational technique determines a cluster or classification of related values.

29. The method of claim 27, wherein the at least one computational technique includes a statistical algorithm.

30. The method of claim 27, wherein the at least one computational technique performs error modeling.

31. A computer-readable medium carrying one or more sequences of instructions from a user of a computer system for visually inspecting diverse, very large data sets of biological data to identify trends, correlations or relations among data from the data sets, wherein the execution of the one or more sequences of instructions by one or more processors cause the one or more processors to perform the steps of:
de-normalizing experimental data contained in an experimental biological data set and supporting data contained in at least one biological supporting data set;
inputting the de-normalized experimental biological data and the de-normalized biological supporting data to the one or more processors;
controlling the processor to generate a matrix containing all of the de-normalized data inputted from the experimental biological data set and each supporting data set, wherein the matrix contains rows pertaining to items upon which experiments were performed, at least one column containing values obtained as a result of the experiments performed on the corresponding items, and at least one column containing supporting data corresponding to the items in the rows;
displaying the matrix, in compressed format, on a display screen such that all of the data is graphically represented on the display screen, wherein the experimental values are expressed graphically in compressed format with the size and direction of the graphical representation indicating the relative value of the experimental values; and wherein adjacent, like values in the supporting data columns are represented by a block, line or other graphical representation;
sorting any selected column of the matrix to arrange the column in an order of ascending or descending values; and
expanding one or more selected rows of the matrix to be displayed in a noncompressed format.

32. The computer readable medium of claim 31, wherein the following further step is performed: overlaying a graphical representation of the experimental data set on the display of the matrix.

33. The computer readable medium of claim 31, wherein the following further step is performed: substantially simultaneously highlighting data in the matrix and data in at least one of the data sets from which the data was inputted to generate the matrix.

34. The computer readable medium of claim 31, wherein the following further step is performed: displaying graphical representations of the experimental data displayed in expanded form, said graphical representations corresponding to graphical representations of the experimental data which are contained in the experimental data set.

35. The computer readable medium of claim 31, wherein the following further steps are performed: monitoring the number of rows included in each said block, line or other graphical representation formed to indicate locations of adjacent, like values in the supporting data columns; and
overlaying a descriptive label over each said block, line or other graphical representation which includes at least a minimal predetermined number of rows, wherein the descriptive label describes a common feature of the data represented by the block, line or other graphical representation.

36. The computer readable medium of claim 27, wherein the following further step is performed: performing at least one computational analysis on at least one column of values of the matrix to determine values for a new column to be added to the matrix, and means for displaying the determined values in the new column in the matrix.
